(19) United States
(12) Patent Application Publication
(10) Pub. No.: US 2013/0041051 A1
(43) Pub. Date: Feb. 14, 2013

(54) METHOD FOR PRODUCING A METHANE-RICH PRODUCT GAS AND REACTOR SYSTEM USABLE FOR THAT PURPOSE

(71) Applicants: Ulrich Zuberbuhler, Stuttgart (DE); Bernd Sturmer, Stuttgart (DE); Volkmar Frick, Stuttgart (DE); Michael Specht, Waldenbuch (DE); Martin Buxbaum, Alberschwende (DE)

(72) Inventors: Ulrich Zuberbuhler, Stuttgart (DE); Bernd Sturmer, Stuttgart (DE); Volkmar Frick, Stuttgart (DE); Michael Specht, Waldenbuch (DE); Martin Buxbaum, Alberschwende (DE)

(73) Assignee: SOLAR FUEL GMBH, Stuttgart (DE)

(21) Appl. No.: 13/517,941

(22) PCT Filed: Nov. 11, 2010
(86) PCT No.: PCT/EP2010/006877

§ 371 Date: Oct. 25, 2012

(30) Foreign Application Priority Data
Dec. 23, 2009 (DE) 10 2009 059 310.1

(51) Int. Cl. C07C 1/12; B01J 19/00

(52) U.S. Cl. 518/712; 422/105

(57) ABSTRACT

The invention relates to a method for producing a methane-rich product gas, in which a starting gas containing hydrogen and carbon dioxide is catalytically methanated under the influence of at least one adjustatable parameter in at least two stages and at least one criterion relating to the composition of the product gas is monitored. The criterion is fulfilled under a condition influencing the method and when the condition changes, a change in the parameter setting that preserves fulfilment of the criterion is affected.

### CROSS REFERENCE TO RELATED APPLICATIONS

The present application is a 35 U.S.C. 371 national phase entry application of International Patent Application No. PCT/EP2010/006877, filed Nov. 11, 2010, which claims priority to German Patent Application No. DE102009059310.1, filed Dec. 23, 2009, the disclosures of which are hereby incorporated by reference in their entirety for all purposes except for those sections, if any, that are inconsistent with this specification.

### TECHNICAL FIELD

The invention relates to a method for producing a methane-rich product gas, in which a starting gas containing hydrogen and carbon dioxide is catalytically methanated under the influence of at least one settable parameter in at least two stages, and at least one criterion relating to the composition of the product gas is monitored, wherein the criterion is fulfilled under a condition influencing the method, as well as to a reactor system that is suitable for this purpose, and to a product gas produced in this manner.

### BACKGROUND

Methanation methods of this type are known. They are carried out, for example, by means of reactor systems which comprise, for example, two solid bed reactors connected one after the other, which are provided with nickel-containing catalysts. A starting gas which contains hydrogen and carbon dioxide in a stoichiometric ratio which is substantially suitable for the methane production, and which, moreover, can additionally comprise proportions of carbon monoxide, methane, other hydrocarbons as well as contaminants in the form of various minor components and inert gases (for example, N₂), is fed, at a predefined temperature and at a predefined pressure, to the first solid bed reactor (first methanation stage), in which the following processes, which in total form CH₄, occur.
1) CO+H₂O [symbol] CO₂+H₂, the so-called water-gas shift reaction
2) CO+3 H₂ [symbol] CH₄+H₂O, the CO methanation, and
3) CO₂+4 H₂ [symbol] CH₄+2 H₂O, the CO₂ methanation.

In this manner, after the first methanation stage, a certain methane proportion is obtained in this gas exiting said stage. For an additional increase in the methane proportion, the second methanation stage, which is arranged downstream, is used. Using known means, water and optionally excess reactants can be removed from the gas exiting the second stage, to obtain a product gas having a more than 90% methane proportion.

To simplify the language, the term gases (product gas, starting gas, . . . ) is used now and below, although it naturally refers gas mixtures consisting of different components.

The methane-rich product gas produced with this method can be used to feed into various uses, as a “synthetic” mineral gas, with an appropriately high methane proportion. Thus, it is possible to provide that a product gas is fed into existing mineral gas networks. For this purpose, the Standard DVGW G 260/262 must be fulfilled, i.e., the proportion of hydrogen in the product gas must be less than 5%, and the proportion of carbon dioxide must be less than 6%. For the use of the methane-rich product gas as a fuel for driving vehicles, DIN 51624 must be fulfilled, i.e., the proportion of inert gas in the gas mixture must be less than 2%, and the proportion of carbon dioxide must be less than 15%. Such a use of the produced fuel gas is disclosed, for example, in DE 10 2008 053 34 A1.

The reactor systems used for the methanation usually work in continuous operation, wherein one or more process parameters that are suitable for the continuously fed starting gas are selected, to generate a product gas that fulfills, for example, the above-indicated standard.

### BRIEF DESCRIPTION OF DRAWINGS

Further details, characteristics and advantages of the invention result from the following description of individual embodiment examples in reference to the attached figures, of which

FIG. 1 shows a diagrammatic representation of a reactor design according to the invention, and

FIG. 2 shows a diagrammatic representation of an additional embodiment.

### DETAILED DESCRIPTION OF EMBODIMENTS

The invention is based on the problem of improving such a method, particularly with a view to increased flexibility.

In terms of process technology, this problem is solved by a variant of the method mentioned in the introduction, which is substantially characterized in that when the condition changes, a change in the parameter setting that preserves fulfillment of the criterion is carried out.

In this manner, it is possible to react in a flexible manner to a change in the process conditions, which may also be brought about intentionally, for example, wherein it is ensured at the same time that the desired product gas quality is preserved.

The criterion which relates to the composition of the product gas can be a single criterion, for example, the methane proportion of the product gases; however, it may also comprise two or more subcriteria, for example, a maximum hydrogen and/or carbon dioxide content.

In a preferred process design, the starting gas is allowed to flow to the first methanation stage, and the condition relates to the feed stream of the starting gas. In this manner, an increased flexibility with regard to the starting gas feed stream is achieved, which may relate to the feed stream quantity per unit of time alone, but which additionally and/or alternatively may also relate to the composition of the starting gas, making it thus possible to react in a flexible manner to the variations of said composition.

Thus it has been noted that, for example for the first variant of the flowing quantity of starting gas per unit of time, even if the composition of the starting gas remains essentially unchanged, a variation in the quantity of starting gas to be reacted has a considerable influence on the methanation process, to which influence one can however react with the solution according to the invention. This is particularly advantageous if the variation is not a one-time disturbance, and, instead, one has to react to repeatedly occurring variations. In particular, one can then intentionally omit a continuous supply of the reactor system with a constant starting gas flow, which further increases the flexibility of the method.

With regard to the provision of the starting gas, the provision of the CO₂ proportion of the starting gas, for example, presents no special problems, since said gas can be obtained, for example, by means of gas scrubbing from the air, and can thus be fed as pure CO₂, or also with a small proportion of CH₄ leakage from the biogas workup, or alternatively in the form of a raw biogas which comprises, for example, an approximately 45% CO₂ proportion with already an approximately 55% methane proportion (besides minor components that are neglected here). The provision of the hydrogen required for the methanation, at the correct stoichiometric ratio, on the other hand is more expensive, because H₂ has to be manufactured first with energy expenditure. This can occur, for example, by generating the hydrogen on site using electrolyzers. Here, the electrolyzer itself represents a load connected to an electrical supply grid, wherein the maximum conversion of said electrolyzer defines a corresponding maximum load. The electrolytically produced hydrogen is mixed prior to the first methanation stage with the carbon dioxide-containing contribution to the starting gas, so that the ongoing load of the electrolyzer substantially also defines the quantity of starting gas to be reacted per unit of time.

In this manner, the methanation method can be used to store regenerative energies that are not generated continuously over time, for example, wind or solar energy. The electrolyzer, in the case of a low regenerative energy generation, and absence of compensation by means of other connected energy sources, collects a correspondingly lower load, which ensures load changes, including rapid load changes, to which it is possible to react using the method according to the invention.

The method is thus designed for an intermittent operation between the normal state and a lower utilized capacity of even 60% or less, preferably 40% or less, and particularly also 25% or less of the maximum utilized capacity. It is possible to switch between these states of lower load and the normal state, for the purpose of increasing the output, in switching times of less than 10 min, preferably less than 4 min, and particularly less than 1 min; the output reduction is possible in half the time in each case.

In a particularly preferred embodiment, an energy expenditure connected with the methanation depends on the setting of the at least one parameter, wherein the setting change which has been made reduces the energy expenditure. Thus, an energy-optimized procedure is achieved while the product quality remains the same, wherein the energy expenditure considered here includes, for example, the energy consumption associated with the compression of gases (compression), or the energy consumption associated with the addition by metering of water and its workup and/or the separation of water from the gas, as well as additional heat outputs, but not the energy consumption used for the electrolysis of the hydrogen used.

In a particularly preferred embodiment, the influence of the at least one parameter acts between the first and the second of the at least two methanation stages. In this manner, the modular construction of the process stages can be used advantageously for the control of the method while still fulfilling the criterion; in particular, this simplifies the energy-optimized process control.

A parameter that is important for this purpose is the pressure, which can be changed according to the invention between the first and the second methanation stage. This changeability, even implemented independently of the remaining process characteristics, is also considered advantageous from the standpoint of the invention. The invention thus also discloses, in the most general form, a method for producing a methane-rich product gas, in which a starting gas containing hydrogen and carbon dioxide is catalytically methanated in at least two stages, method which is substantially characterized in that the pressure of the gas entering into the second stage can be changed with respect to the pressure of the gas exiting the first stage.

In a base setting with steady-state (full) load, at the time of inflow into the first stage, a gas pressure 1 bar or more, and stage of 16 bar or less, preferably 8 bar or less, and particularly 4 bar or less can be set, and, before the second reactor, a gas pressure between 1 and 16 bar, preferably between 3 and 10 bar, and particularly between 5 and 8 bar can be set. Here and also below, pressure refers to absolute pressures.

An additional parameter whose influence acts between the methanation stages is the water content in the gas after the first stage. In this regard, the invention also considers a partial water removal advantageous, even independently of the remaining process characteristics. The invention in its generality thus also discloses a method for producing a methane-rich gas mixture, in which an input gas mixture containing carbon dioxide and hydrogen is catalytically methanated in at least two stages, wherein, in a first methanation stage carried out at a first site, a water-containing intermediate gas mixture forms, which one then allows to flow to a second site for a second methanation stage, and which is characterized substantially in that one removes a first portion of the water contained in the intermediate gas mixture from the flow of the intermediate gas mixture, while one leaves a second portion of the water therein and allows it to continue to flow to the second site.

In the context of the invention, a combination of these two independently disclosed aspects (pressure change and dew point setting between the stages) is also considered advantageous.

In this context, an additional specification is preferred, in which the partial removal of the water is achieved by cooling the intermediate gas mixture to a temperature which is below the dew point, but which is at least 60° C., preferably at least 80° C., particularly at least 86° C., and even more preferably at least 100° C., and/or a specification, according to which the cooling temperature of the intermediate gas mixture is 160° C. or less, preferably 135° C. or less, and particularly 128° C. or less. In this regard, it is provided in particular that the water content of the intermediate gas mixture which is allowed to continue to flow, in mol %, is 20% or more, preferably 25% or more, particularly 30% or more, and/or 70% or less, preferably 50% or less, and particularly 35% or less. A humid gas is preferably compressed. If this cannot be done with a compressor provided for that purpose, a complete condensation must be selected. The water content at the time of inflow into the first stage depends on the CₓH_{y} content at the time of inflow into reactor, and it can be 0-50 mol %, and preferably 0-30 mol %.

When the reactor system for carrying out the method is utilized to full capacity, a state also referred to as the normal state below, the method should preferably be carried out, with regard to additional parameters, using certain base settings which are indicated below.

Desired Temperatures:

The starting gas should be introduced at a temperature in the range from 250° C. to 330° C., preferably 270° C. to 290° C., in the first methanation stage. For this purpose, the starting gas can be preheated, particularly using the waste heat gained due to the exothermic methanation. This prevents the cooling of the inflow area of the stage, and the formation of a resulting inactive zone, as well as, in the case of the use of a Ni-containing catalyst, the risk of a nickel carbonyl formation.

In the first reactor, maximum temperatures in the range from 350° C. to 650° C., preferably 480° C. to 580° C., should occur, wherein alternatively a negative temperature gradient in the flow direction of the gas, caused by an appropriate coolant circulation, can be provided, or the cooling is preferably carried out with concurrent flow. The outlet temperature can be in the range from 280° C. to 400° C., and preferably 300° C. to 350° C.

On the other hand, in the second methanation stage, the maximum temperatures should be lower, in the range from 280° C. to 400° C., preferably 260° C. to 280° C., with inlet temperatures in the range from 250° C. to 350° C., preferably 270° C. to 330° C., and outlet temperatures in the range from 230° C. to 350° C., preferably 270° C. to 300° C.

For the generation of negative temperature gradients in the reactors, the circulation used for cooling the gases is directed in the countercurrent direction with respect to the gas flow; however, it is preferred to use cooling with concurrent flow.

Desired Space Velocities:

The desired space velocities in the first methanation stage are in a range from 2000/h to 12,000/h, preferably in a range from 2000/h to 8000/h. In the second methanation stage, the desired space velocities are in a range from 1000/h to 6000/h, preferably in a range from 1500/h to 4000/h. If the utilized capacity of the reactor system is low, the space velocities are lowered correspondingly. Here and below, all the space velocities are with respect to the dry gas flow rates.

Desired Recirculation:

The ratio of the recirculated gas quantity to the starting gas quantity is preferably in the range from 0 to 5, particularly in the range from 0 to 1.5 in the case of a starting gas consisting substantially only of CO₂ and H₂, and 0 to 0.5 in the case of the use of a biogas as CO₂-containing component of the starting gas.

Desired Methanation Rates:

After the exit from the first stage, a proportion of conversion of CO₂ to CH₄ of 50 to 90 vol %, preferably 70 to 80 vol %, is sought; after the second stage said proportion is at least 75 vol %, preferably at least 90 vol %, particularly at least 93 vol % or also at least 95 vol %.

In reference to Claim 7, the at least one parameter is selected advantageously from a group which comprises: the pressure existing at the time of inflow into the first methanation stage; the pressure existing at the time of inflow into the second methanation stage; the quantity of a gas that has been diverted and recirculated before the first stage; the water content of the gas before the first methanation stage; the water content of the gas before the second methanation stage; a water quantity removed from the gas mixture after the first methanation stage; a heating output used to heat the gas before the first and/or second methanation stage; a YES/NO inclusion of a third methanation stage; the temperature in the first or (separately) in the second methanation stage; and the composition of the starting gas.

Some of these parameters have already been explained above. An additional parameter to be taken into account particularly with a view to the energy optimization is the quantity of the water fed before the individual stages, to the extent required. The possible inclusion of a third methanation stage further increases the flexibility, and it uses the modular construction, which is provided according to the invention, of the reactor design that is the basis of the method.

In a particularly preferred embodiment, the setting of the changes occurs automatically. Thus, for example, it is possible to provide that the method is carried out in an energy saving mode in which the at least one parameter, preferably two or more parameters, are set automatically in an energy-decreasing manner. The energy saving mode can be triggered preferably by a signal which indicates that the change from the first to the second condition has taken place, in other words that the process condition to be taken into account is again considered steady-state. A testing criterion in this regard can be designed so that a signal is triggered as soon as the process condition considered does not change more than to a predefined extent, over a predefined time period. Alternatively, it is also possible to provide that the method is carried out in principle in an energy saving mode, and is interrupted only upon a predefined interruption signal which is triggered, for example, when the method is to be switched from a low utilized capacity to a higher utilized capacity. This is important particularly in the case of the above-described intermittent operation mode.

In this context, the automatic setting change can be implemented via an adjustment. For this purpose, it is preferable to provide a first adjustment circuit for adjusting the pressure change between the first and the second stage. Alternatively, it is additionally preferable to provide a second adjustment circuit for adjusting the recirculated gas quantity. Moreover, the invention provides for a third and fourth adjustment circuit for the adjustment of the water content in the gas before the first/second methanation stage. By means of two or more mutually engaged adjustment circuits, the process control with optimized energy can be further improved.

In some cases it can be advantageous if the setting change is subject to a general condition which is to be maintained. As examples of general conditions the following can be used: maximum temperature of the gas or of the catalyst in the first and/or second methanation stage; minimum and/or maximum water content of the gas before the first and/or second methanation stage; minimum and/or maximum quantity of the recirculated gas; minimum and/or maximum quantity of the water removed after the first methanation stage; minimum and/or maximum quantity of the pressure existing in the first methanation stage; and fulfillment of an additional (second) criterion relating to the composition of the gas mixture produced.

For example, the setting of the pressure before the first methanation stage can be adjusted with the target requirement that the criterion has been fulfilled, wherein the condition that a maximum temperature of the gas in the first methanation is not exceeded is applied to the adjustment as a general condition to be observed. Thus, the general condition can also be incorporated as a second adjustment criterion in an adjustment. An additional advantageous use is the adjustment of the water removal after the first methanation stage, which (co)adjusts the water content before the second methanation stage, and which can be subject to the general condition that a connector arranged downstream is operable for the adjustment of the pressure in the second methanation stage for such a water content.

The controls/adjustments of more than one parameter can also be interrelated according to the invention. For this purpose, the adjustments can take place in parallel and at the same time: however, it is also possible to provide a first adjustment to be carried out depending on a second adjustment, and to vary the latter for different target parameters, according to which an energy-optimized adjustment of the first parameter is carried out for each variation, and the result thereof is compared, after which the setpoint value for the second adjustment that is selected is the one at which the absolute energy minimum for the first parameter can be reached. Such a variant can be used particularly in the case where the setpoint can be predefined in any case only empirically or by means of appropriate models.

In a particularly preferred embodiment of the method, it is provided that the setting change of the at least one parameter, in the case of an increase of the quantity of the starting gas flowing per unit of time, is to be carried out, in a first intervention, in such a manner that the reaction rate is increased. For this purpose, the pressure in particular can be increased, and the quantity of the recirculated gas can be adapted. This intervention is used particularly advantageously in the case where, during an intentional powering up, the energy optimization becomes secondary to a rapid increase in the reactor output. In this case, the above-explained energy saving mode can be switched off, and the process can be switched to a “power up” mode.

Then, it is particularly preferable to carry out a counter measure in a second intervention, as soon as a (negative) general condition is fulfilled, particularly as soon as a predefined temperature has been exceeded in the first methanation stage. In this manner, an effective protection for the individual components, particularly for the catalysts of the reactor system operated using the method, is given precedence over an accelerated stepping up to higher outputs. The counter measure can consist, for example, of a decrease of the pressure in reactor 1, of the increase in the quantity of the recirculated gas and/or of the reactor cooling power, or it may consists at least partially of said measures.

Advantageously, in a third intervention, an energy saving control is then carried out according to one of the previously described aspects, as soon as a steady-state state has been reached, with regard to the changed condition. Here, the settings carried out in the first and second process step are corrected again, without taking into account the energy balance. The method is switched to the energy saving mode.

The changing condition can also relate, alternatively or additionally, to the composition of the starting gas flow, particularly to the carbon dioxide proportion thereof. In this way, it is possible to achieve a product quality that remains the same, even in the case of a varying starting gas composition.

Alternatively or additionally, the condition can advantageously also relate to the wear of at least one of the catalysts used in the methanation stages. Thus, it is possible to counteract an influence that decreases the product gas quality and is due to the creeping, unavoidable decrease in the activity of the catalysts.

The fast reaction to changed conditions with the above-mentioned low adaptation times can also relate to changed requirements for the criterion itself that is to be monitored, for example, if there is to be a switch from the production of a product gas to be fed into a gas network, to a fuel gas to be used as combustion gas for vehicles. In this case, the changed condition can also consist of the implementation of an additional subcriterion.

In other words, the invention also discloses a method for producing a methane-rich gas mixture, in which a starting gas containing hydrogen and carbon dioxide is catalytically methanated, under the influence of at least one settable parameter, in at least two stages, wherein an energy expenditure that is associated with the methanation depends on the setting of the parameter, and wherein the composition of the product gas fulfills a first criterion which is characterized substantially in that, in the case of a change in the criterion to be fulfilled by the product gas to a second criterion, a change in the parameter takes place, which results in the fulfillment of the second criterion, and which in particular is carried out automatically and minimizes the energy expenditure.

In regard to the apparatus technology, the invention provides a reactor system for producing a methane-rich product gas from a starting gas containing hydrogen and carbon dioxide, with at least two successively connected reactor stages comprising a catalyst, a monitoring device for monitoring the criterion relating to the composition of the product gas, and a control device coupled to the monitoring device, and at least one setting device controlled by the control device, for setting at least one parameter influencing the methanation, wherein the reactor system is operable under a first condition that influences the methanation, and the criterion is fulfilled during said operation, which criterion is characterized in particular in that the reactor system is designed for the purpose of allowing, when there is a change of the first condition to a second condition, a change in the set parameter, which preserves fulfillment of the criterion.

The advantages of such a reactor system result from the above explanations of the method.

The at least two reactor stages preferably can differ with regard to their construction. In this manner, on the one hand, a modular reactor construction is achieved, the individual stages of which can nevertheless be designed individually for the essential field of application. In particular, the construction can differ in terms of mechanical configuration, the type of heat removal and/or the type of catalyst used.

Besides the difference in construction, the individual reactor stages can also be operated using different process parameters. For this purpose, it is provided that a property of the gas fed into a respective reactor stage is modifiable, particularly by means of the at least one setting device. This modular construction with individual settability of individual parameters is also predefined independently of the type of control of the reactor, as an independent disclosure.

Furthermore, it is advantageously provided that at least one first setting device is arranged between the two reactor stages, and particularly that it presents a compressor or consists of one. The advantages achieved with the variable settability of the pressure at this site result from the above explanation of the method. In particular, the invention also discloses independently a reactor system with a first reactor stage, a second reactor stage connected behind the first reactor stage, and a device for changing the pressure of the gas exiting the first reactor stage and entering the second reactor stage, which is arranged between the first and the second reactor stages.

Preferably, the compressor is designed in such a manner that it is operable for a water content present in the form of steam in the gas to be compressed of up to 35%, preferably up to 50%, and particularly up to 60%. This creates the possibility of controlling/adjusting a water content adjustment before the second stage solely by means of the water removal between the two reactor stages.

Such a device for removing at least a portion of the water contained in the gas after the first reactor stage is provided, for example, as a second setting device of the reactor system, and it is arranged in particular upstream of the compressor.

Here, the second setting device allows any desired dew point setting; in particular, it is controlled in such a manner that the water content of the portion remaining in the gas after the partial water removal can be between 0% and 50%, preferably between 20% and 50%, and particularly between 25% and 35%.

Moreover, the reactor system preferably comprises a second setting device for feeding water before one or more reactor stages. The third setting device can be divided into an appropriate number of the sub-devices associated with the individual reactor stages.

Moreover, a fourth setting device is advantageously provided for heating the gas introduced into one or more reactor stages, which setting device can also consist in particular of several sub-devices associated with the individual reactor stages. Here, the sub-device which is arranged upstream of the first reactor stage is used to first heat the starting gas to a temperature that allows the reaction start. The gas exiting the first reactor stage as a rule has been at a sufficient temperature for the second reactor stage; however, due to the cooling occurring for the water separation by condensation between the reactor stages, the heating device associated with the second reactor stage is needed to bring the temperature of the gas again to the temperature desired in the second reactor stage. Both heating devices in the form of heat exchangers, for example, receive the energy required for heating their heat carrier preferably from the waste heat generated in the exothermic reaction and removed from the reactor stages.

It is particularly preferred that a recirculation device, as an additional setting device, is provided for recirculation of a portion particularly of the gas exiting the second reactor stage to a site located before the first reactor stage. The use and the resulting advantages of the recirculation device have already been described in reference to the methanation process, including the controllable flow restrictor/compressor which is usable in the recirculation.

In an additional possible embodiment, the reactor system presents a third reactor stage which is connectable via the control device. This design increases the flexibility achieved with the modular construction of the system. In this regard, the invention also provides for implementing a reactor system independently of the precise control, which system has two series connected reactor stages through which gas flows, and an optionally connectable third reactor stage.

To the extent described to date, the reactor system assumes obtaining a starting gas fed via a correspondingly designed line. However, the system can contain a coupled mixing device for mixing individual components of the starting gas, and it can be provided with a corresponding number of feed lines. Here, a feed line can be coupled, and in particular is coupled, to a device for producing hydrogen by means of electrical energy, for example, to one of the electrolyzers already explained above.

With regard to the carbon dioxide component of the starting gas, it is possible to provide that a gas scrubbing device, which is arranged close to the reactor stages, can be coupled, and in particular is coupled, which device can filter carbon dioxide out of the air and feed it into the mixing device. The carbon dioxide can also be separated from a biogas, wherein up to approximately 5% methane content may remain as residue of the biogas in the starting gas.

Moreover, an additional line leading to the mixing device and designed for controllable feed stream of a biogas can be placed, through which line a predefinable portion of a biogas can be fed as a component of the starting gas.

Preferably one or more of the setting devices are controllable via an adjustment circuit. For this purpose, the control device can obtain, from measurement sensors, signal data with the actual values that are required for the adjustment, and can transmit control data corresponding to the adjustment to the respective setting device. The variables that are preferably adjusted and the corresponding setting devices result from the above explanations concerning the process aspects. The control device, in particular also for carrying out a process, is thus operable according to one or more of the explained process aspects.

To that extent the reactor system is also designed for intermittent operation between two gas quantities of different size to be reacted. In this case, the two conditions relate to different quantities of the starting gas reacted per unit of time; in other words, they relate to different utilized capacities of the system, particularly caused by a load change of the coupled electrolyzer.

The invention also protects a methane-rich gas mixture produced according to one of the above explained process aspects, or using a reactor system in one of the above-described embodiments.

With a view to an independent embodiment of the partial water removal between the two methanation stages, it is provided preferably that the two sites are two sequentially arranged reactor stages of a reactor system, which are arranged one after the other, each of which contains a catalyst, and in which the respective methanation step takes place, wherein the catalytic methanation of the input gas mixture introduced into the first reactor stage occurs at temperatures particularly in the range from 300° C. to 600° C., particularly in the case where the intermediate gas mixture that continues to flow is introduced into the second reactor stage, and the second methanation step occurs there at temperatures in the range in particular from 250° C. to 350° C., and particularly up to 300° C., wherein the input gas mixture can in particular have a carbon monoxide content of less than 0.1%.

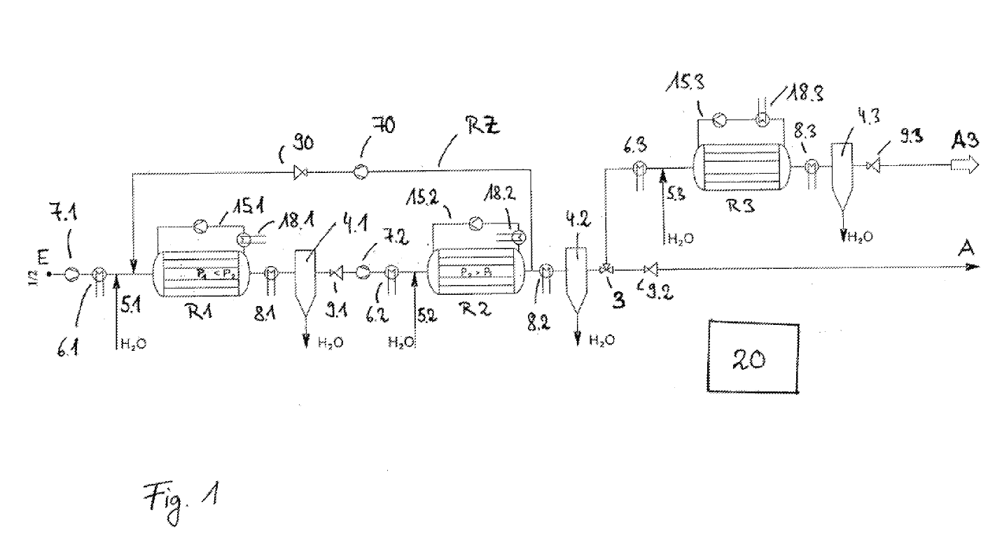

As is evident from the representation of FIG. 1, a starting gas is to be fed into the reactor system at the site marked E, and, if only the reactors R1 and R2 are used, is transferred at the site marked A for further processing, for example, in order to be fed into the existing mineral gas network, or for production in the form of a combustion gas for vehicles of all types.

On the path between the sites E and A, the starting gas flows through two reactors, the reactor R1 and the downstream reactor R2, each if which is provided with a catalyst and has a heat removal system 15.1 or 15.2. The reactors R1 and R2 are designed as solid bed reactors, and they can differ in design and type of catalyst used. It is preferable to use tube bundle reactors or also plate reactors, in order to be able to remove more easily the heat generated in the strongly exothermic methanation. In addition, the steps can be implemented with different process parameters (pressure, temperature, space velocity), for reasons pertaining to the different setting devices used in the reactor design, among other factors.

Thus, first, using a compressor 7.1, the gas pressure for the starting gas is settable before inflow into the reactor R1. Downstream of the compressor 7.1, a heat exchanger 6.1 is arranged, by means of which the starting gas can be brought to a preselectable temperature, for example, 270° C. 5.1 marks an H₂O feed device which is arranged behind the heat exchanger 6.1, and by means of which the water content of the starting gas is settable. A recirculation line RZ opens into the section between the H₂O feed device 5.1 and the inflow into the reactor R1, and, in this embodiment example, said line branches off behind the second reactor R2, and allows a portion of the gas exiting the reactor 2 to be recirculated before the reactor 1.

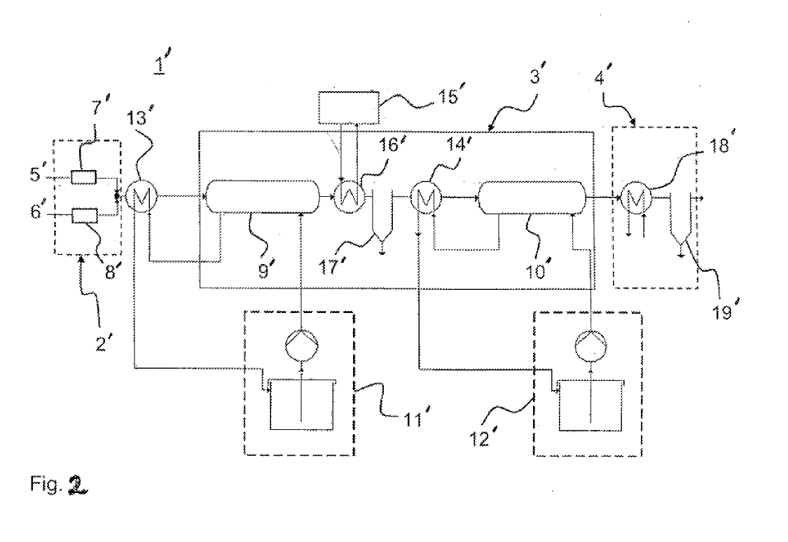

An additional heat exchanger 8.1 is arranged downstream of the reactor 1, by means of which heat exchanger a cooling of the gas exiting the reactor R1 is achievable. A desired dew point setting can be carried out, so that at the site marked 4.1 a proportion of the water contained in the gas, which corresponds to the dew point setting, can be removed. For this purpose, a condensate diverter known from the prior art can be used; see also the more detailed explanations in reference to FIG. 2.

Additional devices up to the inflow of the gas into the second reactor R2 are, in the sequence of the gas flow, a controllable flow restrictor 9.1, an intermediate compressor 7.2, a heat exchanger 6.2 for reheating the gas mixture, as well as an additional H₂O feed device 5.2, by means of which the water content of the gas entering into the reactor R2 can be increased further (again).

In the diagrammatic representation of FIG. 1, the second reactor R2 is diagrammaticaliy represented as identical to the reactor R1; however, in comparison to the reactor R1, it can comprise a catalyst having a different activity, particularly a higher activity, and it can also be designed differently in other respects.

As already explained above, by means of the recirculation line RZ, a portion of the gas exiting the reactor 2 can be recirculated before the reactor 1. In the recirculation line RZ, a controllable flow restrictor 90 is provided, as is a recirculation compressor 70. The latter is optional, and it can also be omitted, because of the pressure control via the intermediate compressor 7.2, which is explained below. The recirculation line RZ may also branch off between the reactors R1 and R2, preferably behind the intermediate compressor 7.2. If this is not the case, however, the recirculation compressor 70 in RZ should be preserved at any rate.

In a manner similar to that used with the reactor R1, for the reactor R2 as well, a cooling device is provided in the form of a heat exchanger 8.2, for the purpose of the dew point setting with appropriate water separation 4.2, and a controllable flow restrictor 9.2, before reaching the outlet site A. However, before the flow restrictor 9.2, a switching device 3 is also built in, by means of which the gas can be diverted to a third reactor R3. The reactor R3, in the embodiment example shown in FIG. 1, completes the stage-like modular construction of the reactor system, and it has, like the reactors R1 and R2, an upstream heating device in the form of a heat exchanger 6.3, and an H₂O feed device 5.3, and downstream it has a cooling device in the form of an additional heat exchanger 8.3 for a renewed dew point setting with appropriate water separation 4.3 and with a controllable flow restrictor 9.3, before reaching the outlet A3, which is active in the case of the use of the third reactor R3. It is also possible to completely omit the third reactor R3, so that the gas exiting the reactor R2 can form the final product gas without further workup measure, with the exception of the subsequent separation of water.

The reactor system is controlled by a control device 20, which by wire connection or wirelessly, transmits its control commands to the individual setting devices, and receives sensor data from the measurement sensors, not shown in FIG. 1, which are explained in further detail below.

First, however, some of the events that occur in the methanation are explained, and measures are indicated to counteract negative effects of said events on the method.

Besides the reactions indicated in equations (1)-(3) which are explained in the introduction, other reactions also occur, such as the conversion of carbon monoxide to elemental carbon which, in part recombined with other elements, is separated on the catalyst surface in the reactor, and can possibly lead to a continuously progressing deactivation of the catalyst. In order to avoid undesired carbon separations, which affect the activity of the catalyst, in the first reactor R1, or at least to reduce them, H₂O in the form of steam can be fed into the starting gas before it enters the reactor 1, provided a sufficient water content has not already been introduced via the recirculation. For this purpose, if needed, the H₂O feed device 5.1 is provided.

The methanation is a strongly exothermic reaction, so that, during the methane formation in the gas, the temperature of the gas also increases strongly (cause of the hot spots). In this manner, one reaches methane contents by the methanation in the first reactor R1 of approximately 65%-85 vol %. To further increase the methane content, a staggered temperature level is implemented in the reactors R1, R2 and optionally R3, which shifts the equilibrium of the reaction to higher methane contents. To preserve these temperature levels, reactor cooling is provided for each reactor step, on the one hand. A cooling circulation 15.1 (15.2, 15.3), which is provided for that purpose, and which is cooled by means of a heat exchanger unit 18.1 (18.2, 18.3) on the reactor side and accordingly absorbs the reactor heat for another utilization, is provided in such a manner that the cooling circulation flow is cocurrent with respect to the reactor gas flow. Alternatively, a temperature gradient that is negative in the direction of the gas flow can also be generated via a countercurrent cooling circulation flow, which is energetically advantageous, with a view to the cooling that occurs subsequently in any case.

For a further improved methanation in the second reactor R2, it is provided that a portion of the water is allowed to condense, in the water removal station by means of dew point setting and water separation by cooling of the gas exiting the reactor R1, which portion corresponds to the set dew point, while an additional portion remains in the gas flow to prevent carbon deposits in the second reactor R2; on this subject see also the basic explanations in reference to FIG. 2.

In particular in the case where the starting gas consists predominantly or substantially of a stoichiometrically appropriate CO₂/H₂ mixture, due to the reaction which already at the time of inflow into the reactor 1 occurs at a very rapid rate at the desired space velocity, a reaction heat is already generated which is so high that a sufficient reactor cooling via the cooling circulation is no longer sufficient. Then, locally delimited superheated zones can form in the inlet area of the reactor, which are referred to as “hot spots.” The formation of such “hot spots” can be detected by the temperature sensors, and corresponding data can be transmitted to the control device 20. Since the catalyst, in the long run, is unable to withstand the temperature stresses in the superheated zones, counter measures are carried out to lower the reaction rate.

A measure that is provided for that purpose is the recirculation RZ of a portion of the substantially already completely reacted product gas exiting, for example, the reactor 2, which is again admixed with the starting gas before the inflow into the first reactor R1. To maintain the pressure level, the recirculated portion can be compressed beforehand by the compressor 70, which compensates for the pressure loss via the reactors R1 and R2. While the reaction rate in the reactor R1 decreases due to this measure, one must in addition ensure that one adapts or possibly stops the H₂O addition via the H₂O feed device 5.1 on the basis of the recirculated methane.

An additional measure to counteract the formation of “hot spots” or to eliminate “hot spots” that have built up consists of further slowing the reaction rate by lowering the pressure of the starting gas before the first reactor R1 via the compressor 7.1. The pressure can be lowered considerably here, and only a slight excess pressure with respect to the environment is required in order to prevent the intake of air in the case of a leak; for example, an absolute pressure of 1.5 bar_{abs} can be selected.

Subsequently, the reaction rate and the local superheated zones regress. By means of the intermediate compressor 7.2, the low pressure in the system can again be powered up for the reactor R2, in which the problem of “hot spots” substantially does not occur. Accordingly, the post-methanation can again be carried out at a higher pressure, for example, 7 bar, which pressure increases the reaction rate due to the volume-reducing reaction, and shifts the equilibrium to higher methane contents. In this case it is even possible for a pressure drop of the pressure behind the reactor R2 to occur, compared to the pressure before the reactor R1, so that the recirculated gas does not have to be compressed again. Accordingly, it is possible to omit the use of the recirculation compressor 70, and the quantity of the optionally humid recirculated gas is set by the controllable flow restrictor 90 alone.

As already explained above, in normal operation (base case), for example, in case of full utilization of the capacity, it is assumed that the product quality is sufficient (fulfillment of the criterion), on the basis of the corresponding base settings, after passing through the two reactors R1 and R2. For the case where, for example, due to a poorer starting gas quality or a decreasing activity of the catalyst, or due to the performance of a load change, the criterion cannot be fulfilled using only the two reactors R1 and R2, the methanation reaction can still be continued by connecting the reactor R3. This occurs by rerouting the gas flow through the rerouting device 3. Since the reactor R3 can be used only for the final methanation, a catalyst presenting maximum activity can be used for that purpose. Furthermore, via the water removal control 8.2 and 4.2, which is connected upstream, a renewed cooling with dew point setting and corresponding water removal is provided, so that only the water content required for the reactor design of the reactor R3 remains in the gas flowing in it, again for the purpose of counteracting undesired carbon deposition on the catalyst.

Taking into consideration the above explained measures, individual controls of the reactor operation are now explained, which optimize, on the one hand, the intermittent operation achieved with the invention (adaptation to a load change). Additional optimizations concern the saving of the energy used for the method as well as a longer utilization of the catalyst.

Only a least possible compression effort is described next as a first aspect. As already explained above, the modular division of the reactor system into the mutually independently settable reactors R1 and R2, by means that include the intermediate compressor 7.2, allows an arbitrarily selectable pressure setting between the individual reactors R1 and R2. With regard to the pressure setting in the reactor 1 via the compressor 7.1, the reactor R1 is operated at a very low pressure (for example, in the range from 1.5 to 2 bar); however, said pressure has to be set to a higher value than the ambient pressure by a predefined safety margin.

Due to the strong volume-reducing reaction in the first reactor, a considerable energy saving is already achieved, since the intermediate compressor 7.2 has to bring only a smaller volume to a desired higher pressure in the reactor 2, smaller than would be the case if a compression were carried out already before the reactor 1. In addition, the pressure in the reactor R2 should also be lowered as much as possible with energy saving, so that the desired quality of the product gas (fulfillment of the first criterion) is still barely reached.

With a fixed pressure in the reactor R1, this energy optimizing pressure lowering for the reactor R2 occurs automatically via an adjustment circuit integrated in the control device 20. As an example of the criterion to be fulfilled, a minimum methane content can be used as target value for the adjustment circuit. The actual value of the adjustment circuit is then the methane content of the product gas, measured at the outlet site. In the case of a positive adjustment deviation between the measured and the required methane content, the pressure before the reactor R2, as control parameter of the adjustment circuit, continues to be lowered until the pressure has been adjusted to a value at which the actual value and the target value are in agreement, and thus the desired product quality is reached with minimum energy expenditure. In the process, the desired criterion can also contain a safety margin with respect to the minimum requirements.

Due to the adjustment circuit, a fitting pressure setting before the reactor 2 occurs independently of whether a disturbance requiring a post adjustment is explained by creeping degradation of one of the catalysts, a slight load change, or variations, for example, in the composition of the carbon dioxide proportion of the starting gas.

An additional parameter that is subject to the optimizing adjustment is the quantity of the recirculated gas, based on the knowledge that the energy expenditure at the intermediate compressor 7.2 rises in accordance with the quantity of the recirculated gas.

This additional adjustment (adjustment circuit 2) is coupled, based on the above explanation of the problems associated with “hot spots,” with the condition that the temperature in the reactor R1 does not exceed a predefined limit temperature which is an experience-based value, with a safety margin, below a catalyst limit temperature, and which is applied as target parameter to the adjustment circuit 2. The temperature measured in the reactor 1 selves as actual value, and the recirculated gas quantity which functions as control parameter is set via the flow restrictor 90 arranged in the recirculation circuit RZ. The recirculated gas quantity is reduced here as long as the measured temperature is lower than the predefined target temperature. Again, in this manner, disturbances which lead to an increase in the temperature existing in the reactor 1, and which possibly increase the risk of the formation of “hot spots,” such as a variation in the starting gas quality, and even a more rapidly reacting composition, or an increase in the starting gas quantity to be reacted per time, are compensated by an automatically adjusted (increased) recirculation.

Via optimization in the form of the smallest possible quantity of the recirculated gas, a highest possible reactor temperature is reached at the same time in the reactor R1, so that the energy value of the usable waste heat, which can be collected via the associated cooling circulation 15.1 and the heat exchanger 18.1, is also maximized.

As already explained above, at each time of inflow into the reactor, the gas should preferably contain a minimum proportion of water, to protect the catalysts against undesired carbon deposition. Besides this protective effect, the feeding of water through the H₂O addition devices 5.1 and 5.2 has a negative effect on the energy balance of the method, since the water has to be worked up, heated, and evaporated, and moreover generates a pressure loss that has to be compensated by the subsequent compression. On the other hand, the fed water, after the respective reactors, is taken out again by condensation, which also increases the process energy.

An additional control of the reactor system therefore ensures that the water proportion is kept as low as possible at each time of inflow into the reactor. With regard to the reactor R1, the water which has been fed in the form of steam through the H₂O water addition device 5.1 should be minimized. An associated limit condition is that the water content in the gas does not fall below a water content in the gas, which depends on several parameters and which is determined using a predefined calculation procedure, at the time of inflow into the reactor R1. For the verification of the fulfillment of this general condition, the gas composition is determined continuously at each time of inflow into the reactor, and transmitted to the control device 20. The parameters by means of which the target water content is calculated consist of one or more of the following: the starting gas composition, particularly the hydrocarbon content including the recirculation in the calculation, the conversion rate particularly of the reactor R1, as well as the pressure and temperature of the reactor R1.

This control can also be carried out automatically by means of an additional adjustment circuit (adjustment circuit 3), wherein the indirectly determined/measured water content of the gas at the reactor inflow is used as actual value, and the quantity of the water, which has been added by metering through the H₂O addition device 5.1, represents the setting parameter of this third adjustment circuit. An indirect determination is obtained from knowing the quantity of the starting gas, the product gas as well as the recirculated gas.

In a similar manner, the water content of the gas entering the reactor R2 should also be kept as low as possible. In comparison to the first reactor R1 there is, however, the difference that the water content is changeable via two measures: on the one hand, via a water removal 4.1 by means of dew point setting by cooling 8.1, and, on the other hand, via water addition by metering through the H₂O water addition device 5.2. Thus, for a corresponding adjustment, two setting parameters are available. Ideally, the feeding of water through the H₂O water addition device 5.2 should be omitted completely, and the adjustment should take place only by dew point setting and water removal after the exit of the gas from reactor R1. However, the latter is subject to the general condition of a maximum permissible water content for the intermediate compressor 7.2. If this water content is exceeded, the adjustment via dew point setting and water separation ends, and the remaining post-adjustment occurs by water addition (5.2). As target value for the water content at the time of the inflow into reactor R2, a value is used again, which is obtained by applying a predefined calculation procedure using the pressure in the reactor R2, the intermediate product gas composition (particularly its hydrocarbon content), and the reactor temperature in the reactor R2.

On the basis of this adjustment circuit 4 and the adjustment circuit 3, a protection of the catalysts is achieved automatically, and an adjustment which counteracts the interfering parameters is carried out, independently of their origin, parameters which otherwise would result in further degradation of the catalysts.

An additional optimization is provided for the case where the criterion to be fulfilled concerns, for example, the methane content of the product gas, but has been made more stringent (or simplified). Besides an adaptation to this changed criterion in order to fulfill it via the pressure in the reactor R2 by means of the intermediate compressor 7.2, the composition of the starting gas can be modified as an additional parameter. This can take place, on the one hand, via a modified mixing ratio of the hydrogen produced, for example, by electrolysis, to the carbon dioxide-containing proportion of the starting gas, or by a variation of the latter proportion. For this purpose, for example, a proportion of biogas can be fed through a connection to an external gas line, biogas which itself already contains a high methane proportion.

An additional particularly important optimization of the reactor operation consists of the control of the latter for intermittent operation. As already explained above, the reactor should not only be usable in continuous operation, it should also allow the processing of, for example, excess currents originating from renewable sources, by means of a feasible load change, within a short time (i.e., by reacting different, rapidly changing, starting gas quantities per unit of time).

First, the reactor system is designed for maximum gas conversion, for which parameters that are reasonable in terms of energy are set in accordance with the above adjustments. By means of such an output, or the load associated with it, the reactor can be operated within a switching time of less than 5 minutes, particularly less than 2 minutes, at a lower load, for example, 40% of the maximum load or less, and particularly 20% of the maximum load or less. It is also possible to use a complete switching off. This load change in general, and rapid load changing in particular, which is exceedingly problematic for conventional solid bed reactors, is facilitated by the modular reactor construction according to the invention as well as the control thereof.

The basic settings for a completely adjusted normal operation can be the following, for example: The starting gas is first preheated to a temperature of 270° C. (6.1). In the first methanation stage, the operation is carried out at temperatures in the range from 270° C. to 550° C. and at a pressure of 2 bar. The cooling, after the passage through the first methanation step, occurs at a temperature between 100° C. and 135° C. (8.1), after which the condensed water proportion is separated (4.1). The subsequent compression (7.2) occurs at a pressure of 7 bar. To the extent that a water content of the gas mixture is still under 20%, it is increased to 25-35% (5.2), wherein a preheating of the recompressed gas to approximately 280° C. is carried out (6.2). The methanation in the second step then occurs at temperatures between 350° C. and 280° C., and at a pressure of 7 bar.

A description is now provided, using a first example, showing how the control of the method takes place in the case where the reactor is to be slowed down from the adjusted normal operation at near maximum output to 20% of said output, for example, because the electrical power obtained from renewable energies for the electrolyzer for hydrogen generation has clearly decreased due to a lull in the wind and/or due to low solar irradiation. On the other hand, it is conceivable that the generation of hydrogen is slowed down because the current required for this purpose is available only at uneconomical prices on the electrical current market.

Then, due to the reduction of the starting gas flow, a reduced space velocity occurs in the reactors, and the load on their catalysts is reduced, allowing even higher methanation degrees. Accordingly, applying the above described adjustments, particularly the adjustment circuits 1 and 2, the criterion of a minimum methanation proportion can also be fulfilled with lower energy expenditure, so that, in case of decreasing output due to the adjustment circuit, corresponding energy savings occur automatically.

In the reverse case, when the control device 20 receives signals to the effect that the reactor system should be powered up from a comparatively low load of, for example, 20% of the maximum load, to full output, the control and adjustment operations directed towards optimizing the energy consumption are first suspended, because there is insufficient energy in the process, and because rapid production of the required reaction heat for the load increase is more important than potential energy savings during the load increase. A more stringent criterion can be predefined in a preparatory and transient manner, for adjustment purposes.

The control device 20, in preparation for the load change, controls one or more of the setting devices, particularly the devices 7.1, 6.1, 7.2, 90, in such a manner that parameters which influence the release of reaction heat are changed to values that promote said release. In this manner, the pressure for reactor R1 and/or the pressure for reactor R2 are/is increased, the recirculation is reduced if necessary, and the starting gas is optionally heated to higher values. Although the more stringent criterion (for example, CH₄ content) may not be fulfilled during the load change, the original criterion is still respected.

A counter measure against the parameter setting which inhibits the reaction rate and the release of the reaction heat starts as soon as excessively high temperatures build up locally in the reactor R1, and “hot spots” form. After a levelling off to the new output (by reaching steady-state reaction conditions under the new load), the control/adjustment with a view to optimizing the energy consumption can be resumed, and the more stringent criterion can optionally be lowered.

An additional possibility for managing a load increase in as short a time as possible consists of the connection of the reactor R3. During the normal operation of the reactor system, which is controlled with energy optimization, this reactor R3 is preferably connected only if the previously explained additional possibilities for maintaining the required product gas quality (fulfillment of the criterion) have already been exhausted. In the representation shown in FIG. 1, it is provided that the reactor R3 can be connected (3), and it can be arranged downstream of the reactors R1 and R2 due to its modular connection. In an alternative embodiment, not shown, of the invention, it has however also been considered to completely omit the reactor R3, and to seek a greater flexibility, optionally by a larger-sized dimensioning of the second reactor R2.

The modular construction of the reactor system from individual reactors which can be mutually coupled in the series system, and which can also be manufactured more simply as parts of a series system in an industrial, cost effective series production, thus allows a greater flexibility in terms of the processing of different starting gases, and the achievement of different output ranges. Moreover, by means of the individual controllability of the individual reactors, particularly by influencing the gas flowing between the two reactors R1 and R2, the explained more flexible control of the system, in particular an energy-optimized control, is made possible.

Below, an additional embodiment is also described, in reference to FIG. 2. The details regarding the dew point setting or the gas mixture described therein can basically also be applied to the embodiment described in reference to FIG. 1, while the parameter values listed below must be interpreted not as limiting the invention, rather as a possible selection or possible embodiment example.

The reactor system 1′ consists of a mixing unit 2′ for mixing carbon dioxide (CO₂) and hydrogen (H₂) in a predetermined molar ratio SN, of a synthesis unit 3′ for the methanation of the carbon dioxide- and hydrogen-containing gas mixture as well as of a drying unit 4′ in which water is removed from the methanated gas mixture in the synthesis unit 3′.

The mixing unit 2′ comprises a feed line 5′ for carbon dioxide (CO₂) and a feed line 6′ for hydrogen (H). In the feed lines 5′, 6′, a mass flow controller (MFC) 7′, 8′ is provided, by means of which the gas flow flowing through the respective feed line 5′, 6′ can be controlled. Downstream of the mass flow controllers 7′, 8′, the two feed lines 5′, 6′ are merged into one gas line.

The synthesis unit consists, as shown in FIG. 2, of two mutually successively arranged reactor stages 9′ and 10′, each of which comprises a nickel-containing catalyst which in itself is known.

The two reactor stages 9′ and 10′ are each cooled from the outside by means of an appropriate cooling medium. The cooling medium used here is led in each case in a circulation along the outer wall of the respective reactor stage 9′, 10′, so that the cooling medium is capable of absorbing a portion of the heat generated by the exothermicity of the methanation reaction (3) from the respective reactor stage 9′, 10′. In the cooling medium circulation of the first and of the second reactor stages 9′ and 10′, a cooling medium temperature maintenance apparatus 11′, 12′ is contained, which brings the cooling medium to a predetermined temperature, before it is led along the outer wall of the reactor stage 9′, 10′.

The cooling agent coming from the cooling medium temperature maintenance apparatus 11′, 12′ is led past the first or past the second reactor stage 9′, 10′, in a direction opposite the flow direction of the gas mixture, i.e., from the outlet of the reactor stage 9′, 10′ to the inlet of the reactor stage 9′, 10′. The result of this is that the cooling effect at the outlet of the reactor stage 9′, 10′ is at a maximum, and decreases continuously toward the inlet of the reactor stage 9′, 10′, because the temperature of the cooling agent increases toward the inlet, due to the heat energy absorbed by the gas mixture, and thus the temperature difference between the temperature of the gas mixture and the temperature of the cooling medium decreases.

The cooling agent, which comes from the reactor stage 9′, 10′, and which has been heated by the exothermicity of the methanation reaction (3), before being returned to the temperature maintenance apparatus 11′, 12′, is first used to preheat, in a heat exchanger 13′, 14′, the gas mixture before the inflow into the first reactor stage 9′ or before the inflow into the second reactor stage 10′. Cooling agent coming from the heat exchanger 13′, 14′ is returned, in the case of the cooling agent circulation of the first and also the cooling agent circulation of the second reactor stage, in each case to the temperature maintenance apparatus 11′, 12′.

Between the two reactor stages 9′, 10′, the synthesis unit 3′ comprises a device for the dew point setting of the gas mixture after exiting the first reactor stage 9′, and before the heat exchanger 14′, for preheating the gas mixture flowing into the second reactor stage 10′. This dew point setting device consists of a cooling agent circulation having a cooling unit 15′ and a heat exchanger 16′ for cooling the gas mixture exiting the first reactor stage 9′, as well as a condensate diverter 17′ which removes the water condensed due to the cooling of the gas mixture from the synthesis system 3′ or from the reactor system 1′.

After the exit from the second reactor stage 10′, the methanated gas mixture is introduced into a drying unit 4′. This drying unit 4′, similarly to the device for the dew point setting, consists of a cooling agent circulation having a cooling unit (not shown), and a heat exchanger 18′ for cooling the gas mixture exiting the second reactor stage 10′, as well as of a condensate diverter 19′ which removes the water which has condensed due to the cooling of the gas mixture from the drying unit 4′ or from the reactor system 1′.

In the mixing unit 2′, by an appropriate control of the mass flow controllers 7′, 8′, pure carbon dioxide (CO₂) obtained, for example, from the air or biogas, and pure hydrogen (H₂) obtained, for example, by electrolysis from water, are mixed stoichiometrically. The gas mixture generated in the mixing unit 2′ thus comprises substantially only carbon dioxide and hydrogen, in the present embodiment.

In this embodiment example, the gas mixture is preheated by preheating in the first heat exchanger 13′ before the first reactor stage 9′ to a temperature of approximately 270° C., before it is introduced into the first reactor stage 9′. After the introduction of the gas mixture which has been preheated in this manner, the exothermicity of the methanation reaction which is started by the catalyst leads to a rapid heating of the gas mixture to a maximum temperature of approximately 450° C. Subsequently, the temperature of the gas mixture decreases toward the outlet of the first reactor stage 9′ to a value of approximately 300° C. After the exit from the first reactor stage 9′, the gas mixture is cooled in the dew point setting device to a temperature of approximately 120° C., and the water that has condensed in the process is removed with the condensate diverter 17′ from the synthesis unit 3′ or from the reactor system 1′. After the dew point setting, the gas mixture is preheated with the heat exchanger 14′ to a temperature of approximately 260° C. The temperature of the gas mixture, after the introduction of the gas mixture into the second reactor stage 10′, increases only slightly to approximately 280° C., and it decreases slightly toward the outlet by approximately 10° C. to a temperature of 270° C.

After the methanation in the first and in the second reactor stage 9′, 10′, the H₂O generated by the methanation reaction is removed from the methanated gas mixture in the drying unit 4′, by cooling the gas mixture in the heat exchanger 18′ to a temperature around approximately 30° C., and by removing the water that has condensed in the process, using the condensate diverter 19′.

The pressure in the reactor system 1′ or in the synthesis unit 3′ is set to 6 bar according to the embodiment example. The mass flows of carbon dioxide gas and hydrogen gas, in the present embodiment example, have been set by means of the mass flow controllers 7′, 8′ in such a manner that the space velocity has a value of 3500/h in the first reactor stage, and a slightly lower value of 2500/h in the second reactor stage.

The molar water content in the gas mixture after dew point setting has been between 30% and 35% in the present embodiment example.

After the drying in the drying unit 4, the molar water content of the gas mixture has a value corresponding to the dew point of 30° C.

The conversion of the carbon dioxide in the first reactor stage 9′ is approximately 95% in the embodiment example: the conversion of the carbon dioxide in the second reactor stage 10′ is slightly more than 90% in the embodiment example, so that in the end the methane content in the methanated gas mixture is approximately 99%.

The resulting gas mixture with high methane content can be used directly as fuel for vehicles, or it can be fed directly into the mineral gas network.

However, variant forms of the reactor system 1′ and the method carried out thereby are also possible according to the invention.

Thus, the reactor system 1 has been described with two reactor stages 9′, 10′. However, it is also possible to provide additional reactor stages, if an even more complete methanation is required.

The methanation of a carbon dioxide- and hydrogen-containing gas is possible not only at 6 bar but also at other pressures. However, a pressure in the range from 2 to 15 bar is preferred. A pressure in the range from 2 to 8 bar is particularly advantageous.

Above, a certain temperature management has been described. Other temperature managements are conceivable. Thus, other temperatures for the preheating of the gas before introduction into the first or second reactor stage 9′, 10′ mixture can be selected. Depending on the catalyst used, the method could also be carried out without preheating the gas mixture before its introduction into the first or second reactor stage 9′, 10′. This would reduce the apparatus costs.

The method has been described in the embodiment example with temperatures in the first reactor stage 9′ from 300° C. to 450° C. In this range, the methanation occurs particularly efficiently. However, it is also possible to carry out the methanation in the first reactor stage at other temperatures, wherein temperatures in the range from 300° C. to 600° C. are selected, at which an efficient methanation is achieved. The post-methanation in the second reactor stage has been described in such a manner that it is carried out at temperatures in the range from 270° C. to 280° C., In this range the post-methanation occurs particularly efficiently. However, it is also possible to carry out the post-methanation at other temperatures, wherein temperatures in the range from 250° C. to 300° C. can offer advantages.

For the dew point setting, the cooling of the gas mixture to a temperature of 120° C. has been described. However, depending on the pressure used, another temperature may be necessary to set the dew point optimally. At pressures between 2 and 15 bar, the temperature to which the gas mixture has to be cooled for the dew point setting, to set a molar water content of 30% to 50%, is in a range from 80° C. to 160° C. At pressures in the preferred range from 2 to 8 bar, this temperature is in a range from 86° C. to 128° C. Advantageously, the dew point setting is carried out in such a manner that the molar water content is in a range from 30% to 50%, particularly preferably from 30% to 35%.

In the above embodiment example, certain space velocities have been described, which are particularly advantageous for an efficient methanation. However, other space velocities are also conceivable; the space velocity of the gas mixture in the first reactor stage 9′ is preferably in a range from 2000/h to 8000/h. A space velocity in a range from 2000/h to 6000/h is particularly advantageous. The space velocity in the second reactor stage 10′ is in a range from 1000/h to 6000/h. A space velocity in the range from 1500/h to 4000/h is particularly advantageous.

In the above indicated embodiment example, as gas mixture containing carbon dioxide and hydrogen, a gas mixture is used which consists substantially only of carbon dioxide and hydrogen. However, the gas mixture used for the methanation can also contain additional components. Optionally, prior to the introduction into the first reactor stage 9′, catalyst poisons, such as sulfur compounds for example, have to be removed. The carbon dioxide- and hydrogen-containing gas mixture can be, for example, a biogas, which in addition to carbon dioxide and hydrogen already contains a certain methane proportion and a carbon monoxide proportion of less than 0.1%. The molar ratio between carbon dioxide and hydrogen can here again be obtained by admixing hydrogen.

Furthermore, in the above-described embodiment example, a stoichiometric mixing of carbon dioxide and hydrogen has been described. However, it is also possible to use a mixing ratio of carbon dioxide and hydrogen that differs therefrom.

Finally, it is described in the above-indicated embodiment example to dry the methanated gas mixture by cooling to 30° C., and by removing the condensed water in the drying unit 4′. The drying of the methanated gas mixture can, however, also be carried out by other known methods. Moreover, the drying can be adapted depending on the specifications for the starting gas mixture, and optionally it can also be completely omitted.

The invention is not limited to the embodiment explained in the detailed description of the figures. Rather, the characteristics of the invention disclosed in the above description as well as in the claims can be essential both individually and also in any combination for implementing the invention in its different embodiments.

1. Method for producing a methane-rich product gas, in which a starting gas containing hydrogen and carbon dioxide is catalytically methanated under the influence of at least one settable parameter in at least two stages (R1, R2), and at least one criterion relating to the composition of the product gas is monitored, wherein the criterion is fulfilled under a condition influencing the method, characterized in that
when the condition changes, a change in the parameter setting that preserves fulfillment of the criterion is carried out.

2. Method according to claim 1, wherein the starting gas is allowed to flow to the first methanation stage, and the condition relates to the feed stream of the starting gas.

3. Method according to claim 2, wherein the condition relates to the quantity of the starting gas flow flowing per unit of time.

4. Method according to claim 3, wherein at least a portion of the hydrogen contained in the starting gas is produced particularly by electrolysis by collecting an electrical load, and the condition relates to the collected load.

5. Method according to claim 1, wherein an energy expenditure, which is associated with the methanation, depends on the setting of the parameter, and the setting change that is carried out reduces the energy expenditure.

6. Method according to claim 1, wherein the influence of the at least one parameter acts between a first and a second of the at least two methanation stages.

7. Method according to claim 1, wherein the at least one parameter is selected from a group consisting of:
the pressure existing at the time of the inflow into the first methanation stage;
the pressure existing at the time of the inflow into the second methanation stage;
the quantity of a gas that has been diverted and recirculated before the first stage;
the water content of the gas before the first methanation stage; the water content of the gas before the second methanation stage;
a water quantity removed after the first methanation stage from the gas mixture; a heating output used to heat the gas before the first and/or second methanation stage;
a YES/NO inclusion of a third methanation stage;
the temperature in the first methanation stage;
separately, the temperature in the second methanation stage; and
the composition of the starting gas.

8. Method according to claim 1, wherein the setting change takes place automatically and particularly via an adjustment.

9. Method according to claim 1, wherein the setting change is subject to a general condition to be respected.

10. Method according to claim 9, wherein a general condition is selected from the group consisting of:
the maximum temperature of the gas and/or the catalyst in the first and/or second methanation stage;
the minimum and/or maximum water content of the gas before the first and/or second methanation stage;
the minimum and/or maximum quantity of the recirculated gas;
the minimum and/or maximum quantity of the water removed after the first methanation stage;
the minimum and/or maximum value of the pressure existing in the first methanation stage; and
the fulfillment of an additional (second) criterion relating to the composition of the gas mixture produced.

11. Method according to claim 10, wherein the fulfillment of the general condition is controlled via an appropriately associated adjustment circuit, and is particularly monitored thereby.

12. Method according to claim 10, wherein the setting of an additional (second) process parameter, which has an effect on the question of the fulfillment of the general condition, is varied while fulfilling the general condition, and, for the change in the setting of the (first) process parameter, a variation thereof is carried out with, in each case, a changed second process parameter, in order to obtain, as a function of said setting, the setting of the (first) process parameter that is associated with the lowest energy expenditure.

13. Method according to claim 3, wherein the influence of the at least one parameter acts on the reaction rate of the methanation, and, in the case of an increase in the quantity of starting gas flowing per unit of time, the change in the setting of the at least one parameter is carried out in a first intervention in such a manner that the reaction rate is increased.

14. Method according to claim 13, wherein, in a second intervention, a counter measure is carried out as soon as a general condition is fulfilled, particularly as soon as a predefined temperature in the first methanation step has been exceeded.

15. Method according to claim 14, wherein, in a third intervention, an energy saving control according to one of claim 5 is carried out, as soon as a steady-state state is reached with regard to the changed condition.

16. Method according to claim 2, wherein the condition alternatively or additionally relates to the composition of the starting gas flow.

17. Method according to claim 1, wherein the condition alternatively or additionally relates to the wear of at least one of the catalysts used in the methanation stages.

18. Method according to claim 1, wherein the condition alternatively or additionally relates to the composition of the product gas, particularly to an adaptation of the monitored criterion.

19. Reactor system for producing a methane-rich product gas from a starting gas containing hydrogen and carbon dioxide, with
at least two reactor stages which comprise a catalyst and are connected one after the other,
a monitoring device for monitoring a criterion relating to the composition of the product gas,
a control device coupled to the monitoring device, and
at least one setting device controlled by the control device, for setting at least one parameter influencing the methanation, wherein the reactor system is operable under a first condition which influences the methanation, and the criterion is fulfilled during this operation,
characterized in that the reactor system is designed so that, when the first condition is changed to a second condition, it allows a change in the set parameter that preserves fulfillment of the criterion.

20. Reactor system according to claim 19, wherein the at least two reactor stages differ in their construction.

21. Reactor system according to claim 20, wherein a difference in the construction consists in the mechanical design, the type of heat removal and/or the type of catalyst used.

22. Reactor system according to claim 19, wherein a property of the gas fed a into a respective reactor stage is changeable, particularly via the at least one setting device.

23. Reactor system according to claim 22, wherein at least one first setting device is arranged between the two reactor stages, and particularly comprises and/or consists of a compressor.

24. Reactor system according to claim 19, in which a second setting device is a device, which is arranged particularly upstream of the compressor, for the purpose of removing at least a portion of the water contained in the gas after the first reactor stage.

25. Reactor system according to claim 24, wherein the second setting device allows a preselectable dew point setting.

26. Reactor system according to claim 19, wherein a third setting device is provided for feeding water before one or more of the reactor stages.

27. Reactor system according to claim 19, wherein a fourth setting device is provided for heating the gas introduced in one or more reactor stages.

28. Reactor system according to claim 19, wherein, as an additional setting device, a recirculation device comprises, for the recirculation of a portion of the gas, in particular of the gas exiting the second reactor stage, to a site located before the first reactor stage.

29. Reactor system according to claim 28, wherein the recirculation device comprises a compressor and/or a controllable flow restrictor.

30. Reactor system according to claim 19, which furthermore comprises a third reactor stage which is connectable via the control device.

31. Reactor system according to claim 19, which can be coupled and in particular is coupled to a device for generating hydrogen by means of electrical energy.

32. Reactor system according to claim 19, which can be coupled and in particular is coupled to a device for producing carbon dioxide, particularly via a gas scrubber.

33. Reactor system according to claim 19, which comprises a connection for feeding a gas, particularly a biogas, used as a portion of the starting gas.

34. Reactor system according to claim 19, wherein one or more of the setting devices is controllable via an adjustment circuit.

35. Reactor system according to claim 19, wherein the control device is operable by carrying out a method according to one of claim 1.

36. Reactor system according to claim 19, wherein the two conditions relate to different quantities of the reacted starting gas per unit of time, which system is designed for an intermittent operation between the two gas quantities to be reacted.

37. Methane-rich gas mixture, produced according to a method according to claim 1, or with a reactor system according to one of claim 19.
